(19) United States
(12) Patent Application Publication
(10) Pub. No.: US 2002/0090405 A1
(43) Pub. Date: Jul. 11, 2002

(54) Components of canola for the treatment of cancer

(76) Inventors: Najla Guthrie, London (CA); Elzbieta Maria Kurowska, London (CA)

Correspondence Address: DAVIDSON, DAVIDSON & KAPPEL, LLC, 485 SEVENTH AVENUE, 14TH FLOOR, NEW YORK, NY, 10018, US

(21) Appl. No.: 09/952,478

(22) Filed: Sep. 14, 2001

Related U.S. Application Data
(60) Provisional application No. 60/232,933, filed on Sep. 15, 2000.

(51) Int. Cl. A61K 35/78; A61K 31/355; A61K 31/192; A61K 31/7024; A61K 31/56

(52) U.S. Cl. 424/755; 514/23; 514/171; 514/458; 514/568

(57) ABSTRACT

Disclosed is a pharmaceutical composition comprising at least one canola extract effective in inhibiting cell proliferation in at least one form of cancer and a pharmaceutically acceptable excipient. Also disclosed are methods of use thereof.

This application claims priority from U.S. Provisional Application No. 60/232,933, filed Sep. 15, 2000, hereby incorporated by reference.

### FIELD OF THE INVENTION

The present invention is directed to pharmaceutical compositions and methods for the prevention and treatment of neoplastic and oncogenic disorders with canola extracts.

### BACKGROUND OF THE INVENTION

Cancer is a disease of inappropriate tissue accumulation. Chemotherapeutic agents share one characteristic: they are usually more effective in killing or damaging malignant cells than normal cells. However the fact that they do harm normal cells indicates their potential for toxicity. Animal tumor investigations and human clinical trials have shown that drug combinations produce higher rates of objective response and longer survival than single agents. Combination drug therapy is therefore, the basis for most chemotherapy employed at present (DeVita, V. T. et al., 1995, Cancer 35:98).

Cancer treatment requires inhibitions of a variety of factors including tumor cell proliferation, metastatic dissemination of cancer cells to other parts of the body, invasion, tumor-induced neovascularization, and enhancement of host immunological responses and cytotoxicity. Conventional cancer chemotherapeutic agents have often been selected on the basis of their cytotoxicity to tumor cells. However, some anticancer agents have adverse effects on the patients immune system. Thus, it would be greatly advantageous if a cancer therapy or treatment could be developed that would afford non-cytotoxic protection against factors that might lead to progression of tumors. By virtue of the present invention, it has been discovered that canola extracts can be utilized to inhibit the proliferation of cancer cells.

Canola is a cruciferous crop which is mainly utilized for its extracted oil. After the oil has been extracted a protein rich meal remains which is used as a ruminant in animal diets. Further extraction of the canola meal yields minor components from canola, including, glucosinolates, phenolic acid esters and phenolic acids. The total content of selected minor components in Canola extracts are listed below:

| | μM/g extract | mg/g extract |
|---|---|---|
| Progoitrin | 8.52 | 3.45 |
| Gluconapin | 5.89 | 2.29 |
| 4-hydroxybrassicin | 3.22 | 1.55 |
| Glucobrassicanapin | 0.90 | 0.36 |
| Glucoalyssin | 0.64 | 0.27 |
| Napoleiferin | 0.54 | 0.23 |
| Glucobrassicin | 0.40 | 0.19 |
| Glucoraphanin | 0.22 | 0.09 |
| Sinigrine | 0.19 | 0.07 |
| Gluconasturtin | 0.19 | 0.08 |
| Neoglucobrassicin | 0.06 | 0.03 |
| 4-methoxyglucobrassicin | traces | — |

Glucosinolates present in the extract from flaked, cooked canola seeds are listed below:

| | mg/g extract | % content |
|---|---|---|
| Total glucosinolates | 8.61 | 0.9% |
| (flaked, cooked Canola | | |
| seeds) | | |
| Total phenolic acids | 134.00 | 13.4% |
| (flaked, cooked Canola | | |
| seeds) | | |
| Total phenolic acids | 53.15 | 5.3% |
| (Canola meal) | | |
| Free phenolic acids | 246.64 | 24.7% |
| (Canola meal extract after | | |
| hydrolysis) | | |

*The remaining components of extracts are mostly sugars and small amounts and saponins

Content of phenolic acids in the extract from canola meal (mg/g extract) are listed below:

| | Protocatechuic | Caffeic | p-coumaric | Ferulic | Sinapic |
|---|---|---|---|---|---|
| Free | Trace | 0.03 | 0.02 | 0.02 | 1.03 |
| phenolic | | | | | |
| acids | | | | | |
| Phenolic | Trace | 0.07 | 0.08 | 0.56 | 50.75 |
| acids | | | | | |
| liberated | | | | | |
| from soluble | | | | | |
| esters | | | | | |
| Phenolic | — | Trace | 0.06 | 0.01 | 0.52 |
| acids | | | | | |
| liberated | | | | | |
| from soluble | | | | | |
| glycosides | | | | | |

Content of free phenolic acids in the extract from canola meal after hydrolysis (ng/g extract) are listed below:

| Protocatechuic | Caffeic | p-coumaric | Ferulic | Sinapic |
|---|---|---|---|---|
| Trace | 0.11 | 0.81 | 3.64 | 242.08 |

Content of phenolic acids in flaked, cooked canola seeds (mg/g extract are listed below:

| | Protocatechuic | Caffeic | p-coumaric | Ferulic | Sinapic |
|---|---|---|---|---|---|
| Free | Trace | Trace | Trace | 0.02 | 1.18 |
| phenolic | | | | | |
| acids | | | | | |
| Phenolic | Trace | 0.01 | 0.07 | 0.52 | 131.95 |
| acids | | | | | |
| liberated | | | | | |
| from soluble | | | | | |
| esters | | | | | |
| Phenolic | — | Trace | Trace | Trace | 0.25 |
| acids | | | | | |
| liberated | | | | | |
| from soluble | | | | | |
| glycosides | | | | | |

### SUMMARY OF THE INVENTION

It is an object of the invention to provide a pharmaceutical composition comprising canola extracts.

It is a further object of the invention to provide a pharmaceutical composition comprising canola extracts for the prevention and treatment of neoplastic and oncogenic disorders.

It is a further object of the invention to provide a pharmaceutical composition comprising canola extracts and at least one additional chemotherapeutic agent for the prevention and treatment of neoplastic and oncogenic disorders.

It is a further object of the invention to provide a method for the prevention and treatment of neoplastic and oncogenic disorders in a human patient by administering a canola extract.

It is a further object of the invention to provide a method for the prevention and treatment of neoplastic and oncogenic disorders in a human patient by administering a canola extract and at least one additional chemotherapeutic agent before, during or after the administration of the canola extract.

It is a further object of the invention to provide a pharmaceutical composition comprising rapeseed extracts for the prevention and treatment of neoplastic and oncogenic disorders and methods thereof.

### DETAILED DESCRIPTION OF THE INVENTION

The present invention is directed to a pharmaceutical composition comprising at least one canola extract effective in inhibiting cell proliferation in at least one form of cancer and a pharmaceutically acceptable excipient.

In preferred embodiments, the canola extract is selected from the group consisting of a total phenolic, a phenolic acid, a carotenoid , a tocopherol/sterol, a glucosinolate, and combinations thereof. In certain embodiments, the combination is a glucosinolate and a phenolic.

The canola extract is incorporated into the formulation in an amount to provide a concentration effective to provide an anti-proliferative effect. The concentration can be, e.g. from about 0.01 μg/ml to about 10000 μg/ml. This range is not meant to be limiting as one skilled in the art would be able to determine the effective concentration range to provide the desired effect. The invention is intended to cover any concentration of at least one canola extract which exhibits an anti-proliferative effect on cancer cells.

In certain embodiments, the composition of canola extract comprises a dose of tocopherol/sterol to provide, e.g., a concentration of the tocopherol/sterol from about 0.1 μg/ml to about 500 μg/ml, about 25 μg/ml to about 250 μg/ml or from about 100 μg/ml to about 200 μg/ml.

In certain embodiments, the composition of canola extract comprises a dose of phenolic acids to provide, e.g., a concentration from about 0.1 μg/ml to about 1000 μg/ml, from about 125 μg/ml to about 600 μg/ml, from about 250 μg/ml to about 600 μg/ml or from about 400 μg/ml to about 600 μg/ml.

In certain embodiments, the composition of canola extract comprises a dose of glucosinolate/phenolics to provide, e.g., a concentration from about 0.1 μg/ml to about 1000 μg/ml, from about 10 μg/ml to about 600 μg/ml, from about 150 μg/ml to about 600 μg/ml; or from about 300 μg/ml to about 600 μg/ml.

In certain embodiments, the composition of canola extract comprises a dose of sinapic acid to provide a concentration, e.g., from about 1 μg/ml to about 500 μg/ml; from about 10 μg/ml to about 400; or from about 40 μg/ml to about 200 μg/ml.

In embodiments where the canola extract comprises a glucosinolate, the glucosinolate can be selected from the group consisting of progoitrin, sinigrin, glucoraphanin, napoleferin, glucoalyssin, gluconapin, 4-hydroxybrassicin, glucobrassicanapin, glucobrassicin, gluconasturtin, 4-methoxy-glucobrassicin, neoglucobrassicin and combinations thereof.

In certain embodiments, the pharmaceutical compositions of the present invention inhibit cell proliferation of at least one form of cancer from about 25% to about 100%, preferably from about 50% to about 100% and most preferably from about 75% to about 100%.

The invention is further directed to methods of treating a mammal (e.g. a human patient) at risk of or suffering from cancer comprising administering a canola extract effective to inhibit the proliferation of at least one form of cancer. Preferably, the extract is in the form of a pharmaceutical composition as disclosed herein.

Cancers that can be prevented and/or treated by the compositions and methods of the present invention include colon carcinoma, pancreatic cancer, breast cancer, ovarian cancer, prostate cancer, fibrosarcoma, myxosarcoma, liposarcoma, chondrosarcoma, osteogenic sarcoma, chondroma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

The present invention can be administered intravenously, intraperitoneally, subcutaneously, intramuscularly, intrathecally, orally, sublingually, into the buccal cavity, rectally, topically or by aerosol.

Formulations suitable for oral administration include liquid solutions of the active compound dissolved in diluents such as saline, water or PEG 400; capsules or tablets, each containing a predetermined amount of the active agent as solid, granules or gelatin; suspensions in an approximate medium; and emulsions.

Formulations suitable for parenteral administration include aqueous and non-aqueous isotonic sterile solutions, which contain buffers, antioxidants and preservatives. The formulations may be in unit dose or multi-dose sealed containers.

Formulations suitable for topical administration include creams which contain at least one canola extract alone or in combination with at least one additional chemotherapeutic agent.

Dosage amount and interval may be adjusted individually to provide plasma levels of the canola extract which are sufficient to maintain the anti-proliferative and anti-metastatic effects.

Alternatively, one may administer the compound in a local, rather than oral manner, for example, via injection of the compound directly into a tumor, often in a depot or sustained release formulation.

A variety of delivery systems for the pharmacological compounds may be employed, including, but not limited to, liposomes and emulsions. The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Furthermore, one may administer the agent in a targeted drug delivery system, for example, in a liposome coated with tumor-specific antibody. The liposomes will be targeted to and taken up selectively by the tumor.

In cases of local administration or selective uptake, the effective local concentration of the drug may not be related to plasma concentration.

### BRIEF DESCRIPTION OF THE DRAWINGS

FIG. 1 is a graph depicting cell proliferation (percent of control) versus the medium concentration of phenolics/glucosinolates in cancer cells.

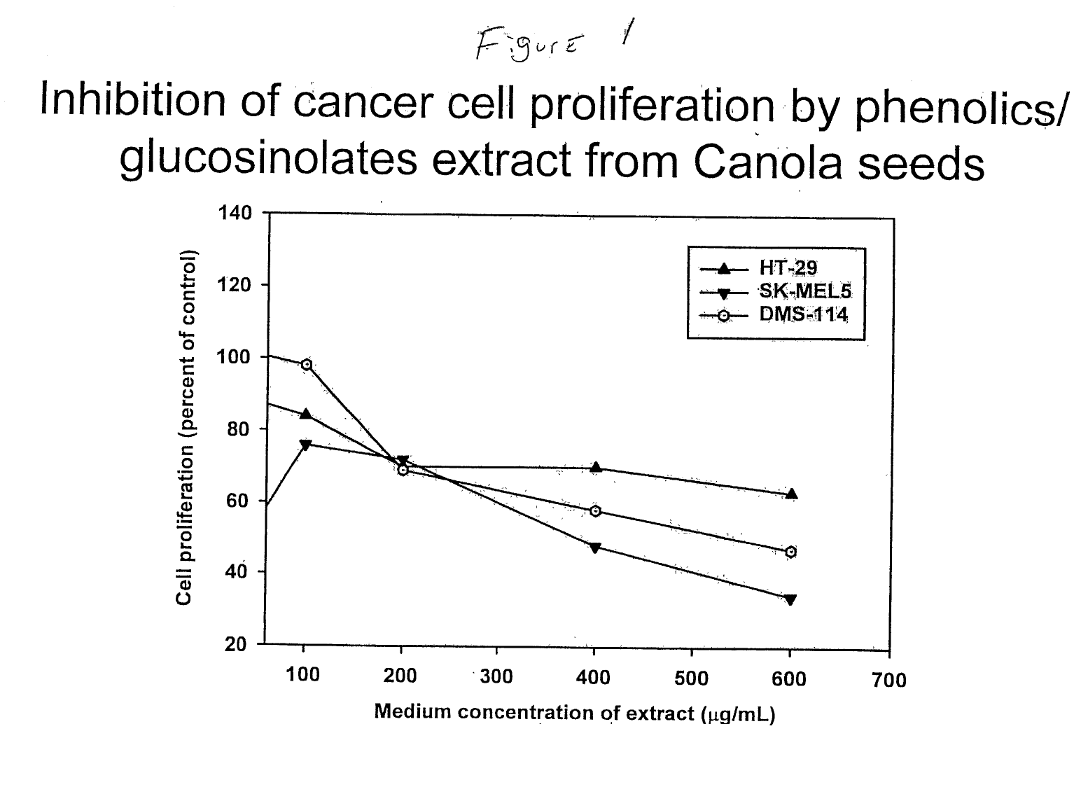

FIG. 2 is a graph depicting cell proliferation (percent of control) versus the medium concentration of phenolic acids.

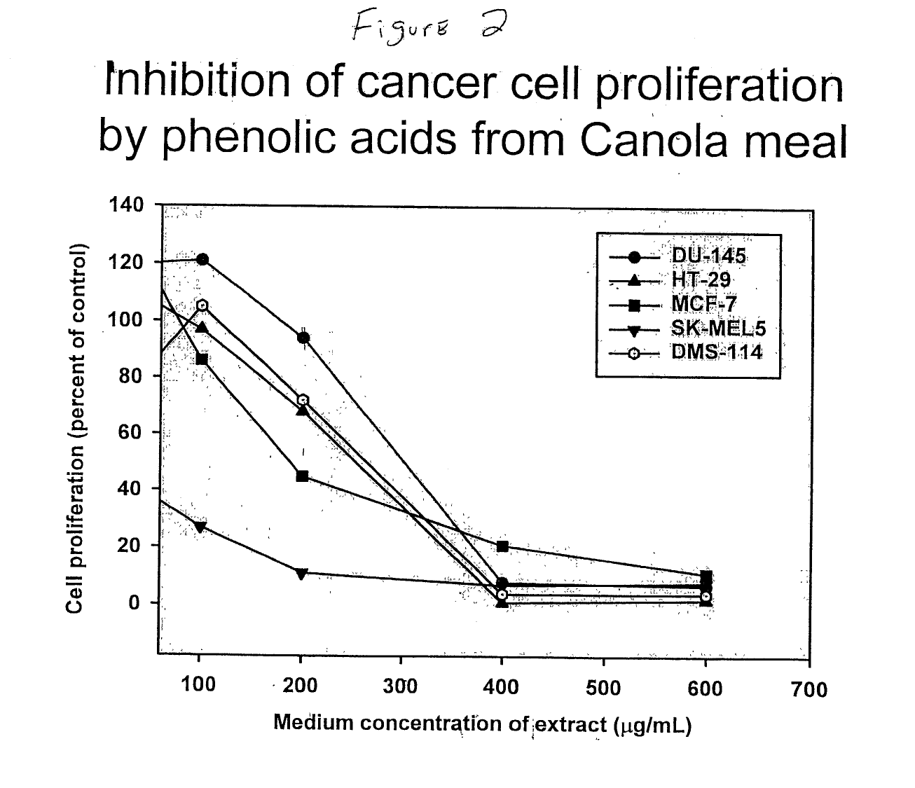

FIG. 3 is a graph depicting cell proliferation (percent of control) versus the medium concentration of tocopherol/sterol.

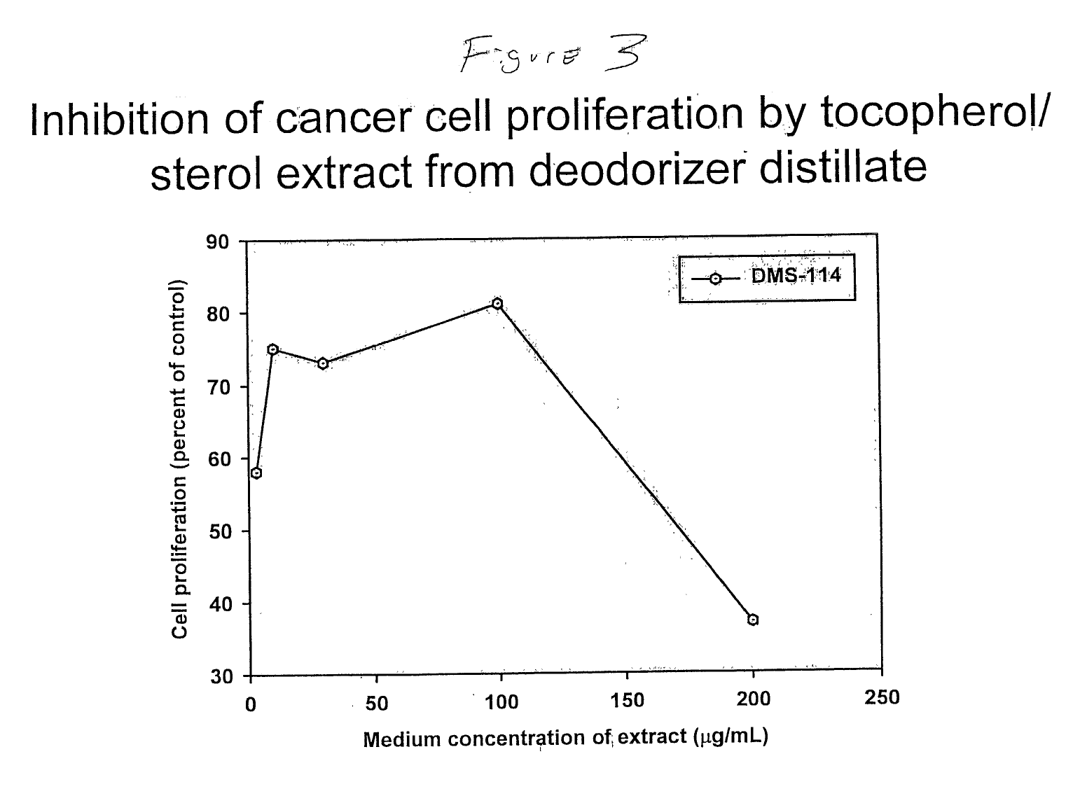

FIG. 4 shows the effects of a sinapic acid containing canola extract and rapeseed extracts on three different cell lines.

### EXAMPLES

A. Extraction of Total Phenolics from Canola Meal

A 2 g sample of canola meal were homogenized in room temp. with 20 mL methanol/water (70:30, v/v). The sample was centrifuged for 10 min, 5,000 rpm and supernatant was collected. The precipitate was extracted 2 more times with fresh portions of methanol/water (70:30, v/v). Combined supernatants were evaporated at 40-45° C. under vacuum and then under nitrogen gas to dryness. For experiments, stock solution (50 mg/mL) was made in DMSO.

B. Extraction of Phenolic Acids from Canola Meal

A 1 g sample of canola meal was homogenized with 20 mL methanol-acetone-water solvent system (7:7:6, v/v/v) for 15 seconds, 10,000 rpm. Extraction was repeated two more times using fresh 1 g samples of canola meal. Combined samples were centrifuged for 15 min, 5,000 rpm. Supernatant was collected and precipitate was extracted 2 more times with fresh portions of methanol/acetone/water. Combined supernatants (˜120 mL from both extractions) were evaporated first at 40-45° C. under vacuum and then under nitrogen gas until the volume reached approximately 20 mL. At this point, 15 mL 4 N NaOH was added and the solution was flushed with nitrogen gas for 4 hours at room temperature. The solution was then acidified to pH 2 using 6 N HCl, transferred to a large saponification tube and extracted 3 x by shaking virgously for 30 seconds with fresh 20 mL portions of diethyl ether-ethyl acetate (1:1, v/v). Combined ether extracts were evaporated to dryness under nitrogen gas. For experiments, stock solution (50 mg/mL) was made in DMSO.

C. Extraction of Carotenoids from Spent Bleach Clay

2 g sample of spent bleach clay was homogenized with 20 mL hexane:acetone:petroleum ether (2:1:1, v/v) at room temp for 1 min and centrifuged for 10 min, 5,000 rpm. Supernatant was collected and precipitate was extracted one more time using a fresh portion of the solvent system. Combined supernatants were protected from light and evaporated under nitrogen gas until a stable volume was reached. For experiments, stock solution (200 mg/mL) was made in DMSO. The highest concentration of carotenoid fraction used was 2mg extract/mL medium (=0.8% DMSO). To determine whether the extract contained carotenoids, a TLC plate was developed in heptane-benzene (9:1, v/v) along with pure carotenoids (beta-carotene or mixture) as standards. Tested sample produced yellow spots at Rf 0.2-0.3, confirming presence of carotenoids.

D. Extraction of Tocopherols/Sterols from Deodorizer Distillate

A 2 g sample of deodorizer distillate was homogenized at room temperature with 20 mL methanol for 1 min and centrifuged for 10 min, 5,000 rpm. Extraction was repeated with fresh portion of hot methanol (to extract sterols). Combined supernatants were evaporated to dryness under nitrogen gas. For experiments, stock solution (200 mg/mL) was made in DMSO.

E. Extraction of Total Phenolics and Glucosinolates from Flaked, Cooked Canola

A 2 g sample of flaked, cooked canola seeds was homogenized at room temp for 1 min. with 6 mL methanol/water (90:10, v/v) and centrifuged for 10 min, 5,000 rpm. Extraction was repeated one more time with a fresh portion of methanol/water. Combined supernatants were evaporated to dryness under nitrogen gas. For experiments, stock solution (70 mg/mL) was made in DMSO.

F. Screening of Extracts Against Cancer Cells

Extracts were screened against human prostatic tumor cell lines (DU 145); human colon cancer cell lines (HT29), human lung cancer cell lines (DMS 114), human melanoma cell lines (SK-MEL-5) and estrogen receptor-positive human breast cancer cell lines (MCF-7). The results are set forth below:

### EXAMPLE 1

Fraction #1 (total phenolics) dissolved in DMSO (1.2% for 600 μg/mL, 0.25% for 125,μg/mL)

| | | |
|---|---|---|
| Conc. | DU-145 | HT-29 |

| μg/mL | Control | + compound | Control | + compound |
|---|---|---|---|---|
| 600/600 | 56.3 | 104.0 | 49.5 | 86.6 |
| 200/125 | 92.9 | 94.8 | 99.9 | 71.5 |
| 60/37.5 | 93.0 | 98.8 | 88.0 | 84.6 |
| 20/12.5 | 99.6 | 91.4 | 88.8 | 79.8 |
| 6/3.75 | 101.0 | 81.9 | 81.4 | 86.1 |

| | | |
|---|---|---|
| MCF-7 | SK-MEL5 (to 3.75) | DMS-114 (to 3.75) |

| Conc. | | + com- | | + com- | | + com- |
|---|---|---|---|---|---|---|
| μg/mL | Control | pound | Control | pound | Control | pound |
| 600/600 | 28.9 | 72.5 | 36.3 | 68.2 | 26.8 | 60.5 |
| 200/125 | 76.1 | 97.0 | 93.4 | 64.0 | 93.5 | 99.5 |
| 60/37.5 | 75.9 | 97.5 | 74.2 | 106.0 | 99.5 | 104.0 |
| 20/12.5 | 77.3 | 90.1 | 84.2 | 82.5 | 101.0 | 96.9 |
| 6/3.75 | 72.9 | 92.0 | 77.5 | 97.1 | 101.0 | 108.0 |

### EXAMPLE 2

Fraction #2 (phenolic acids) dissolved in DMSO (1.2% for 600 μg/mL, 0.25% for 125 μg/mL)

| | | |
|---|---|---|
| Conc. | DU-145 | HT-29 |

| μg/mL | Control | + compound | Control | + compound |
|---|---|---|---|---|
| 600/600 | 56.3 | −10.0 | 49.5 | 13.1 |
| 200/125 | 92.9 | 103.0 | 99.9 | 79.3 |
| 60/37.5 | 93.0 | 109.0 | 88.0 | 107.0 |
| 20/12.5 | 99.6 | 102.0 | 88.8 | 106.0 |
| 6/3.75 | 101.0 | 93.3 | 81.4 | 106.0 |

| | MCF-7 | | SK-MEL5 | | DMS-114 | |
|---|---|---|---|---|---|---|
| Conc. | | + com- | | + com- | | + com- |
| μg/mL | Control | pound | Control | pound | Control | pound |
| 600/600 | 28.9 | −39.0 | 36.3 | −8.5 | 26.8 | −15.0 |
| 200/125 | 76.1 | 75.1 | 93.4 | 17.4 | 93.5 | 97.2 |
| 60/37.5 | 75.9 | 116.0 | 74.2 | 70.7 | 99.5 | 106.0 |
| 20/12.5 | 77.3 | 109.0 | 84.2 | 84.6 | 101.0 | 96.5 |
| 6/3.75 | 72.9 | 111.0 | 77.5 | 113.0 | 101.0 | 89.9 |

### EXAMPLE 3

Fraction #3 (Carotenoids) dissolved on DMSO (0.36% for 0.8% fraction dilution, 0.075% for 0.3% fraction dilution)

| Conc. | DU-145 | | HT-29 | |
|---|---|---|---|---|
| μg/mL | Control | + compound | Control | + compound |
| 0.80/0.3 | 92.9 | 87.2 | 99.9 | 88.3 |
| 0.24/0.1 | 93.0 | 102.0 | 88.0 | 123.0 |
| 0.08/0.03 | 99.6 | 98.3 | 88.8 | 109.0 |
| 0.024/0.01 | 101.0 | 93.8 | 81.4 | 113.0 |
| 0.008/0.003 | 97.3 | 90.8 | 75.6 | 110.0 |

| | | | SK-MEL5 | | DMS-114 | |
|---|---|---|---|---|---|---|
| | MCF-7 | | (to 0.003) | | (to 0.003) | |
| Conc. | | + com- | | + com- | | + com- |
| μg/mL | Control | pound | Control | pound | Control | pound |
| 0.80/0.3 | 76.1 | 36.8 | 36.3 | 67.7 | 26.8 | 97.0 |
| 0.24/0.1 | 75.9 | 93.7 | 93.4 | 68.4 | 93.5 | 108.0 |
| 0.08/0.03 | 77.3 | 99.3 | 74.2 | 70.0 | 99.5 | 109.0 |
| 0.024/0.01 | 72.9 | 113.0 | 84.2 | 96.8 | 101.0 | 97.9 |
| 0.008/0.003 | 93.6 | 93.3 | 77.5 | 109.0 | 101.0 | — |

### EXAMPLE 4

Fraction #4 (Tocopherols/sterols) dissolved in ethanol (0.1% ethanol for conc. 200 μg/mL)

| | | |
|---|---|---|
| Conc. | DU-145 | HT-29 |

| μg/mL | Control | + compound | Control | + compound |
|---|---|---|---|---|
| 200 | 86.0 | 82.0 | 80.2 | 73.2 |
| 100 | 85.8 | 93.3 | 78.4 | 86.0 |
| 10 | 85.8 | 99.4 | 78.4 | 86.8 |
| 30 | 84.6 | 98.7 | 75.1 | 83.1 |
| 3 | 84.6 | 96.9 | 75.1 | 83.4 |

| | MCF-7 | | SK-MEL5 (to 0.003) | | DMS-114 (to 0.003) | |
|---|---|---|---|---|---|---|
| Conc. | | + com- | | + com- | | + com- |
| μg/mL | Control | pound | Control | pound | Control | pound |
| 200 | 82.0 | 91.0 | 97.0 | 86.4 | 85.2 | 31.1 |
| 100 | 77.5 | 115.0 | 90.4 | 107.0 | 87.3 | 70.7 |
| 10 | 77.5 | 114.0 | 90.4 | 96.4 | 87.3 | 65.3 |
| 30 | 73.8 | 108.0 | 89.1 | 96.8 | 102.0 | 74.2 |
| 3 | 73.8 | 92.3 | 89.1 | 94.4 | 102.0 | 59.6 |

### EXAMPLE 5

Compound #5 (phenolics +glucosinates) dissolved in DMSO (1.2% for 600 μg/mL, 0.25% for 125 μg/mL)

| | | |
|---|---|---|
| Conc. | DU-145 | HT-29 |

| μg/mL | Control | + compound | Control | + compound |
|---|---|---|---|---|
| 600/600 | 56.3 | 63.3 | 49.5 | 73.6 |
| 200/175 | 92.9 | 97.7 | 99.9 | 81.4 |
| 60/52.5 | 93.0 | 105.0 | 88.0 | 91.9 |
| 20/17.5 | 99.6 | 87.2 | 88.8 | 91.1 |
| 6/5.25 | 101.0 | 89.6 | 81.4 | 95.7 |

| | MCF-7 | | SK-MEL5 | | DMS-114 | |
|---|---|---|---|---|---|---|
| Conc. | | + com- | | + com- | | + com- |
| μg/mL | Control | pound | Control | pound | Control | pound |
| 600/600 | 28.9 | 21.5 | 36.3 | 26.9 | 26.8 | −11.0 |
| 200/175 | 76.1 | 86.3 | 93.4 | 20.6 | 93.5 | 93.5 |
| 60/52.5 | 75.9 | 93.5 | 74.2 | 62.2 | 99.5 | 103.0 |
| 20/17.5 | 77.3 | 95.3 | 84.2 | 58.8 | 101.0 | 98.1 |
| 6/5.25 | 72.9 | 85.7 | 77.5 | 77.6 | 101.0 | 87.0 |

### EXAMPLE 6

Canola extract (total phenolics isolated from canola meal after hydrolysis, containing 24.7% of free phenolic acids, mainly sinapic acid) and two extracts enriched in free phenolic acids were isolated from rapeseed. Rapeseed extract #1 contained 33.3% sinapic acid equivalents and rapeseed extract #2 contained 61.4% sinapic acid equivalents. Both rapeseed extracts were screened for anti-cancer potential in five human cancer cell lines and the results were compared to those obtained for the canola extract of this example.

Table 1 below shows the effect of rapeseed extract #1 and the canola extract on proliferation of different cancer cell lines.

TABLE 1
Percent proliferation of cancer cells by extracts containing
comparable levels of sinapic acid equivalents.

| | Canola Extract* | Rapeseed Extract #1** |
|---|---|---|
| DU-145 cells | 8% | 22% |
| HT-29 cells | 1% | 30% |
| MCF-7 cells | 21% | 10% |
| DMS-114 cells | 4% | 8% |

*400 mg crude extract/mL medium, containing 99.8 mg/mL of sinapic acid equivalents
**300 mg crude extract/mL medium, containing 98.6 mg/mL of sinapic acid equivalents

As demonstrated in Table 1, both the canola extract and rapeseed extract #1 effectively inhibited proliferation of cancer cell lines when added to culture medium at comparable concentrations of sinapic acid equivalents. In DU-145 cells and in HT-29 cells, the canola extract tended to be more active than rapeseed extract #1.

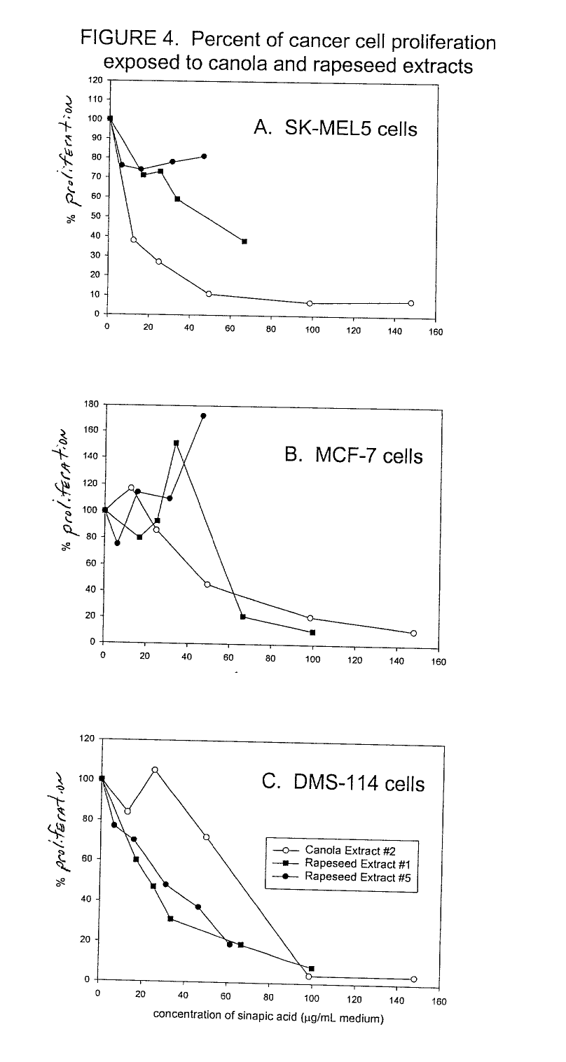

The anti-proliferative activities of canola and rapeseed extracts were also evaluated in human cancer cell lines SK-MEL-5, MCF-7 and DMS-114 at wide range of sinapic acid concentrations. As demonstrated in FIG. 4, the canola extract had greater ability to inhibit proliferation of SK-MEL-5 cells and MCF-7 cells than rapeseed extracts #1 and #2. In contrast, greater activity of rapeseed-based extracts was observed in DMS-114 cells. In all three cell lines, rapeseed extract #2, containing 66.4% sinapic acid, was less active than extract #1 containing 33.3% sinapic acid. This suggests that sinapic acid alone is unlikely responsible for the substantial anti-cancer activity observed for the canola extract.

What is claimed is:

1. A pharmaceutical composition comprising at least one canola extract effective in inhibiting cell proliferation in at least one form of cancer and a pharmaceutically acceptable excipient.

2. The composition of claim 1, wherein the canola extract is selected from the group consisting of a total phenolic, a phenolic acid, a carotenoid, a tocopherol/sterol, a glucosinolate and combinations thereof.

3. The composition of claim 1, wherein the canola extract comprises a tocopherol/sterol.

4. The composition of claim 1, wherein the canola extract comprises a glucosinolate and phenolics.

5. The composition of claim 1, wherein the canola extract comprises a phenolic acid.

6. The composition of claim 1, wherein the canola extract comprises a glucosinolate.

7. The composition of claim 1, wherein the canola extract comprises sinapic acid.

8. The composition of claim 1, wherein the canola extract comprises a glucosinolate selected from the group consisting of progoitrin, sinigrin, glucoraphanin, napoleferin, glucoalyssin, gluconapin, 4-hydroxybrassicin, glucobrassicanapin, glucobrassicin, gluconasturtin, 4-methoxy-glucobrassicin, neoglucobrassicin and combinations thereof.

9. The composition of claim 1, which inhibits cell proliferation of at least one form of cancer from about 25% to about 100%.

10. The composition of claim 1, which inhibits cell proliferation of at least one form of cancer from about 50% to about 100%.

11. The composition of claim 1, which inhibits cell proliferation of at least one form of cancer from about 75% to about 100%.

12. The composition of claim 5, wherein said composition contains a dose of said phenolic acids to provide a concentration of from about 125 μg/ml to about 600 μg/ml.

13. The composition of claim 5, wherein said composition contains a dose of said phenolic acids to provide a concentration of from about 250 μg/ml to about 600 μg/ml.

14. The composition of claim 5, wherein said composition contains a dose of said phenolic acids to provide a concentration of from about 400 μg/ml to about 600 μg/ml.

15. The composition of claim 3, wherein said composition contains a dose of said tocopherol/sterol to provide a concentration from about 25 μg/ml to about 250 μg/ml.

16. The composition of claim 3, wherein said composition contains a dose of said tocopherol/sterol to provide a concentration of from about 100 μg/ml to about 200 μg/ml.

17. The composition of claim 4, wherein said composition contains a dose of said glucosinolate/phenolics to provide a concentration from about 10 μg/ml to about 600 μg/ml.

18. The composition of claim 4, wherein said composition contains a dose of said glucosinolate/phenolics to provide a concentration of from about 150 μg/ml to about 600 μg/ml.

19. The composition of claim 4, wherein said composition contains a dose of said glucosinolate/phenolics to provide a concentration of from about 300 μg/ml to about 600 μg/ml.

20. The composition of claim 7, wherein said composition contains a dose of said sinapic acid to provide a concentration from about 1 μg/ml to about 500 μg/ml.

21. The composition of claim 7, wherein said composition contains a dose of said sinapic acid to provide a concentration of from about 10 μg/ml to about 400 μg/ml.

22. The composition of claim 7, wherein said composition contains a dose of said sinapic acid to provide a concentration of from about 40 μg/ml to about 200 μg/ml.

23. The composition of claim 1 wherein said cancer is selected from the group consisting of colon carcinoma, pancreatic cancer, breast cancer, ovarian cancer, prostate cancer, fibrosarcoma, myxosarcoma, liposarcoma, chondrosarcoma, osteogenic sarcoma, chondroma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblstoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

24. The composition of claim 1, wherein said composition is suitable for intralesional, intraperitoneal, intramuscular, intravenous, infusion;
liposome-mediated delivery; topical, nasal, oral, anal, subcutaneous, vaginal, sublingual, uretheral, transdermal, intrathecal, ocular or otic administration.

25. The composition of claim 1 further comprising at least one additional chemotherapeutic agent.

26. A method of treating a human at risk of or suffering from cancer comprising administering a pharmaceutical composition comprising at least one canola extract effective in inhibiting cell proliferation in at least one form of cancer and a pharmaceutically acceptable excipient.

27. The method of claim 26 wherein said canola extract comprises phenolic acids to provide a concentration of from about 125 μg/ml to about 600 μg/ml at the site of the cancer.

28. The method of claim 26 wherein said canola extract comprises phenolic acids provide a concentration of from about 250 μg/ml to about 600 μg/ml at the site of the cancer.

29. The method of claim 26 wherein said canola extract comprises phenolic acids provide a concentration of from about 400 μg/ml to about 600 μg/ml at the site of the cancer.

30. The method of claim 26 wherein said canola extract comprises tocopherol/sterol to provide a concentration from about 25 μg/ml to about 250 μg/ml at the site of the cancer.

31. The method of claim 26 wherein said canola extract comprises tocopherol/sterol provides a concentration of from about 100 μg/ml to about 200 μg/ml at the site of the cancer.

32. The method of claim 26 wherein said canola extract comprises glucosinolate/phenolics to provide a concentration from about 10 μg/ml to about 600 μg/ml at the site of the cancer.

33. The method of claim 26 wherein said canola extract comprises glucosinolate/phenolics to provide a concentration of from about 150 μg/ml to about 600 μg/ml at the site of the cancer.

34. The method of claim 26 wherein said canola extract comprises glucosinolate/phenolics to provide a concentration of from about 300 μg/ml to about 600 μg/ml at the site of the cancer.

35. The method of claim 26 wherein said canola extract comprises of said sinapic acid to provide a concentration from about 1 μg/ml to about 500 μg/ml.

36. The method of claim 26 wherein said canola extract comprises sinapic acid to provide a concentration of from about 10 μg/ml to about 400 μg/ml.

37. The method of claim 26 wherein said canola extract comprises sinapic acid to provide a concentration of from about 40 μg/ml to about 200 μg/ml.
